(19) United States
(12) Patent Application Publication
(10) Pub. No.: US 2020/0205719 A1
(43) Pub. Date: Jul. 2, 2020

(54) METHOD AND SYSTEM FOR ACTION MONITORING OF RECIPROCATING SPORT

(71) Applicant: Industrial Technology Research Institute, Hsinchu (TW)

(72) Inventors: Yi-Cheng Lu, Hsinchu City (TW); Yun-Yi Huang, Pingtung County (TW)

(73) Assignee: Industrial Technology Research Institute, Hsinchu (TW)

(21) Appl. No.: 16/420,180

(22) Filed: May 23, 2019

(30) Foreign Application Priority Data
Dec. 28, 2018 (TW) 107147566

(51) Int. Cl. A61B 5/22; A61B 5/0488; A63B 24/00; A61B 5/103; A61B 5/00; G16H 20/30; G09B 19/00

(52) U.S. Cl. CPC A61B 5/224; A61B 5/0488; A63B 24/0062; A61B 5/222; A61B 5/1038; A61B 5/1126; A61B 5/7278; G16H 20/30; G09B 19/0038; A61B 2503/10; A61B 5/6829

(57) ABSTRACT

A method and a system for action monitoring of reciprocating sport are provided. The monitoring method is adapted for a monitoring system including a calculation apparatus, at least one gravity sensor disposed on at least one motion part of a human body and at least one electromyography sensor disposed on at least one muscle part of the human body. The method includes: in a process of a reciprocating sport involving multiple actions performed by the human body, sensing a relative angle of each motion part with respect to a reference position of the human body by the gravity sensor and sensing a startup sequence of the muscle part by the electromyography sensor; determining the action performed by the human body according to the relative angle; and determining whether a force applied by the human body is correct according to the action and the startup sequence of the muscle part.

### CROSS REFERENCE TO RELATED APPLICATION

This application claims the priority benefit of Taiwan application serial no. 107147566, filed on Dec. 28, 2018. The entirety of the above-mentioned patent application is hereby incorporated by reference herein.

### TECHNICAL FIELD

The disclosure relates to a method and a system for action monitoring and also relates to a method and a system for action monitoring of a reciprocating sport.

### BACKGROUND

As people today are more and more enthusiastic about exercising, cycling, jogging, mountain-climbing, stepping and hiking are all quite popular sports. Nevertheless, when a user does the aforementioned sports, incorrect movements usually cause discoordination in actions of a human body, resulting in poor efficiency or failure in speed enhancement, and moreover, wrong force applying manners may be likely to cause sports injuries, such as strains, contusions or fractures, to people.

At present, products available on the market measure a size and a power of a force applied by the user during exercise through a strain gauge, however, it is impossible to know whether the actions and the force applying manner when the user is exercising are correct. Thus, how to monitor the correctness and the coordination of the user's actions and force applying manner while the user is exercising has become an important subject.

### SUMMARY

A method for action monitoring of a reciprocating sport of an embodiment of the disclosure is adapted for a monitoring system including a calculation apparatus, at least one gravity sensor (G sensor) and at least one electromyography sensor (EMG sensor), wherein the G sensor is disposed on at least one motion part of a human body, and the at least one EMG sensor is disposed on at least one muscle part of the human body. The method includes the following steps. In a process of a reciprocating sport involving multiple actions performed by the human body, a relative angle of each motion part with respect to a reference position of the human body is sensed by the at least one G sensor, and a startup sequence of the at least one muscle part is sensed by the at least one EMG sensor. The actions performed by the human body are determined according to the sensed relative angle of each motion part. Whether a force applied by the human body to perform the actions is correct is determined according to the actions and the startup sequence of the at least one muscle part.

A system for action monitoring of a reciprocating sport provided by an embodiment of the disclosure includes at least one G sensor, at least one EMG sensor and a calculation apparatus. The at least one G sensor is disposed on at least one motion part of a human body. The at least one EMG sensor is disposed on at least one muscle part of the human body. The calculation apparatus communicates with the at least one G sensor and the at least one EMG sensor and, in a process of a reciprocating sport involving multiple actions performed by the human body, is configured to: sense a relative angle of each motion part with respect to a reference position of the human body by the at least one G sensor and sense a startup sequence of the at least one muscle part by the at least one EMG sensor, determine the actions performed by the human body according to the sensed relative angle of each motion part and determine whether the force applied by the human body to perform the actions is correct according to the actions and the startup sequence of the at least one muscle part.

Several exemplary embodiments accompanied with figures are described in detail below to further describe the disclosure in details.

### BRIEF DESCRIPTION OF THE DRAWINGS

The accompanying drawings are included to provide further understanding, and are incorporated in and constitute a part of this specification. The drawings illustrate exemplary embodiments and, together with the description, serve to explain the principles of the disclosure.

FIG. 1A is a block diagram illustrating an action monitoring system of a reciprocating sport according to an embodiment of the disclosure.

FIG. 1B is a flowchart illustrating a method for action monitoring of a reciprocating sport according to an embodiment of the disclosure.

FIG. 2A is a flowchart illustrating a method for action monitoring of a reciprocating sport according to another embodiment of the disclosure.

FIG. 2B is a diagram illustrating a relationship between the user's stepping action and lifting action and the crank angle according to the embodiment depicted in FIG. 2A of the disclosure.

FIG. 3 illustrates an example of a reciprocating sport done by the human body according to another embodiment of the disclosure.

FIG. 4A is a flowchart illustrating a method for action monitoring of a reciprocating sport according to another embodiment of the disclosure.

FIG. 4B illustrates an example of estimating the crank angle that one of the feet of the human body steps on the pedal of the pedaling apparatus according to the embodiment depicted in FIG. 4A of the disclosure.

FIG. 5A is a flowchart illustrating a method for action monitoring of a reciprocating sport according to another embodiment of the disclosure.

FIG. 5B through FIG. 5I illustrate examples that the human body does the reciprocating sport according to the embodiment depicted in FIG. 5A of the disclosure.

FIG. 6 is a flowchart illustrating a method for action monitoring of a reciprocating sport according to another embodiment of the disclosure.

### DETAILED DESCRIPTION OF DISCLOSED EMBODIMENTS

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

The term “coupling/coupled” used in this specification (including claims) may refer to any direct or indirect connection means. For example, “a first device is coupled to a second device” should be interpreted as “the first device is directly connected to the second device” or “the first device is indirectly connected to the second device through other devices or connection means.” Moreover, wherever appropriate in the drawings and embodiments, elements/components/steps with the same reference numerals represent the same or similar parts. Elements/components/steps with the same reference numerals or names in different embodiments may be cross-referenced.

The embodiments of the disclosure provide a method and a system for action monitoring of a reciprocating sport, capable of monitoring coordination between actions of a reciprocating sport performed by a human body and a muscle startup sequence to monitor whether a force applying manner of the human body is correct, so as to improve an efficiency and a speed for exercising. In this method, information related to a human body of a user doing a reciprocating sport is monitored by using various sensors, which include at least one G sensor for sensing a relative angle of each motion part of the user with respect to a reference position of the human body and at least one EMG sensor for sensing a startup sequence of each muscle part of the user. These information may be integrated to determine whether the actions currently performed by the user are correct and whether a force applying manner to perform the actions is correct. The method of the embodiments of the disclosure can be applied to monitor actions of sports, such as cycling, jogging, mountain-climbing, stepping, hiking, and embodiments are provided below for illustration.

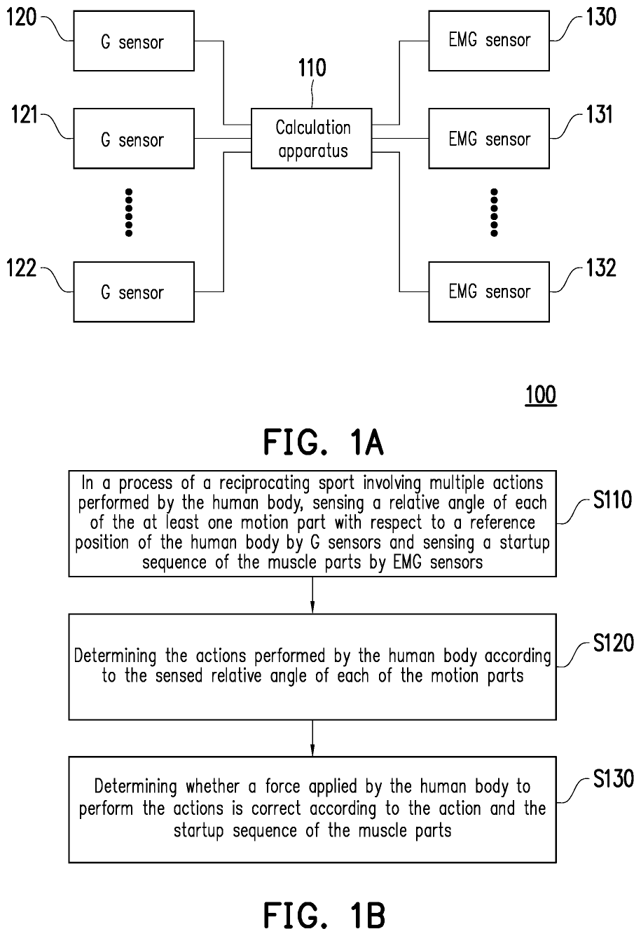

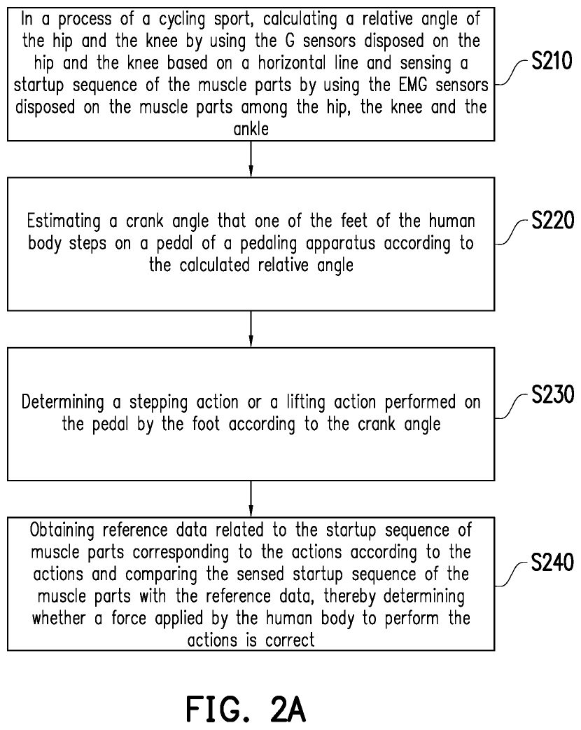

FIG. 1A is a block diagram illustrating an action monitoring system of a reciprocating sport according to an embodiment of the disclosure. Referring to FIG. 1A, an action monitoring system 100 of the present embodiment includes a calculation apparatus 110, at least one gravity sensor (G sensor) 120-122 and at least one electromyography sensor (EMG sensor) 130-132. The G sensors 120-122 are disposed on at least one motion part of a human body, and taking a reciprocating stepping sport for example, the motion parts may include a knee, an ankle and a foot (including a foot heel and a foot sole). The EMG sensors 130-132 are disposed on at least one muscle part of the human body, and taking leg muscles for example, the muscle parts may include a quadriceps muscle, a biceps femoris muscle, a gastrocnemius muscle, a tibialis muscle, a soleus muscle, a rectus femoris muscle, a gluteus maximus muscle, etc. The calculation apparatus 110 is connected respectively with the G sensors 120-122 and the EMG sensors 130-132. It is to be mentioned that to simplify the description, only the three G sensors 120-122 and the three EMG sensors 130-132 are illustrated as being included in the action monitoring system 100 of the present embodiment in FIG. 1, for example, and people with ordinary skills in the art may adaptively adjust the numbers of the G sensors and the EMG sensors according to actual application scenarios, which are not particularly limited in the present embodiment.

The EMG sensors 130-132 and the G sensors 120-122 may be, for example, wearable devices which may be implemented, for example, in a form of patches, straps, waist supports, knee pads, ankle supports, belts, pants, socks, or shoes that may be worn or put on by a user, but the disclosure is not limited thereto. In an embodiment, the calculation apparatus 110 may be a smart device such as a cell phone, a tablet computer, a wristband, a watch, or eyeglasses. In other embodiments, the calculation apparatus 110 may also be disposed on a device (for example, disposed on a bike) which is rode or used for the reciprocating sport, but the disclosure is not limited thereto.

The EMG sensors 130-132 and the G sensors 120-122 are respectively connected with the calculation apparatus 110 through connection devices (not shown) in a wired or a wireless manner. Regarding the wired connection manner, the connection device may be a universal serial bus (USB), an RS232 adaptor, a universal asynchronous receiver/transmitter (UART), an inter-integrated circuit (I2C), a serial peripheral interface (SPI), a display port, a thunderbolt port or a local area network (LAN) interface, which is not limited in the disclosure. Regarding the wireless connection manner, the connection device may be a wireless fidelity (Wi-Fi) module, a radio frequency identification (RFID) module, a Bluetooth module, an infrared (IR) module, a near-field communication (NFC) module or a device-to-device (D2D) module, which is also not limited in the disclosure.

The calculation apparatus 110 may include, for example, a storage device and a processor (not shown). The storage device may be, for example, a random access memory (RAM), a read-only memory (ROM), a flash memory, a hard disk of any type, a like element or a combination of the aforementioned elements. The processor may be, for example, a central processing unit (CPU) or any other programmable general purpose or special purpose microprocessor, digital signal processor (DSP), programmable controller, application specific integrated circuit (ASIC), a like device or a combination of the above devices. In the present embodiment, the processor may load a computer program from the storage device to execute a method for action monitoring of a reciprocating sport provided by the embodiments of the disclosure.

FIG. 1B is a flowchart illustrating a method for action monitoring of a reciprocating sport according to an embodiment of the disclosure. Referring to FIG. 1A and FIG. 1B simultaneously, the method of the present embodiment is adapted for the action monitoring system 100 illustrated in FIG. 1A, and detailed steps of the method for action monitoring of the reciprocating sport of the embodiments of the disclosure is described below in conjunction with the operational relationship between each element of the action monitoring system 100.

First, in step S110, in a process of a reciprocating sport (for example, cycling, jogging, mountain-climbing, stepping, hiking, etc.) involving multiple actions performed by a human body, the calculation apparatus 110 may sense a relative angle of each motion part of the human body with respect to a reference position of the human body by using the G sensors 120-122 and sense a startup sequence of muscle parts of the human body by using the EMG sensors 130-132. Then, in step S120, the calculation apparatus 110 may determine the actions currently performed by the human body according to the sensed relative angle of each motion part with respect to the reference position of the human body. The calculation apparatus 110 may, for example, based on a horizontal line, calculate a relative angle of the knee with respect to the hip of the human body or calculate a relative angle of the ankle with respect to the knee of the human body, which is not limited herein. The calculation apparatus 110 may obtain reference data related to a startup sequence of muscle parts corresponding to the actions according to the determined actions. The reference data may be, for example, a correct startup sequence of the muscle parts related to the actions which are previously stored in a storage device (or a remote server). Finally, in step S130, the calculation apparatus 110 may compare the sensed startup sequence of the muscle parts with the startup sequence of the muscle parts which is recorded in the obtained reference data, thereby determining whether a force applied by the human body to perform the actions is correct. In this way, the present embodiment is capable of monitoring correctness of the startup sequence of the muscles for the human body in the process of the reciprocating sport and thereby, an efficiency of the user doing the reciprocating sport may be improved.

In an embodiment, the calculation apparatus 110, for example, may store information related to angle changes of the motion parts implementing each action and correct startup sequences of the muscles (i.e., the aforementioned reference data) in various types of reciprocating sports in its own storage device. Thereby, the calculation apparatus 110 is capable of, according to the sensed relative angle of each motion part, determining the actions currently performed by the human body by looking up the aforementioned information to find out the correct startup sequence of the muscle parts implementing the actions to compare it with the currently sensed startup sequence of the muscle parts, so as to determine whether the force applied by the human body to perform the actions is correct.

In another embodiment, the action monitoring system 100 may further include a remote server (not shown). The remote server is, for example, a cloud storage device or a cloud server, which stores, for example, the information related to the angle changes of the motion parts implementing each action and the correct startup sequences of the motion parts in various types of reciprocating sports. Thereby, the calculation apparatus 110 may communicate with the remote server through a network, so as to look up the aforementioned information in the remote server to determine the actions currently performed by the human body and find out the correct startup sequence of the muscle parts to compare it with the currently sensed startup sequence of the muscle parts, so as to determine whether the force applied by the human body to perform the actions is correct. The aforementioned network may be, for example, a local area network (LAN) or Internet, but the disclosure is not limited thereto.

In yet another embodiment, besides storing the aforementioned information, the remote server is further capable of determining whether the current actions and the applied force of the human body are correct. In detail, the calculation apparatus 110, may communicate with the remote server, for example, through the network to transmit the sensed relative angle of each motion part with respect to the human body and the startup sequence of the muscle parts to the remote server and receive a determination result about whether the applied force is correct from the remote server. In detail, the remote server, for example, determines the actions currently performed by the human body according to the information received by the calculation apparatus 110 and looks up the correct startup sequence of the muscle parts to compare it with the startup sequence of the muscle parts received from the calculation apparatus 110, so as to determine whether the force applied by the human body to perform the actions is correct and finally, returns the determination result to the calculation apparatus 110.

In light of the foregoing, the action monitoring system 100 of the embodiments of the disclosure may determine whether the force applied by the human body to perform the actions is correct via the calculation apparatus 110 itself, determine whether the force applied by the human body to perform the actions with the assist of the remote server, or directly determine whether the force applied by the human body to perform the actions is correct via the remote server, which is not particularly limited in the disclosure.

Additionally, in an embodiment, the calculation apparatus 110 includes, for example, an alarm device, such as a display, a speaker, a light-emitting diode (LED) array or a vibrator or an arbitrary combination of the aforementioned devices, thereby visually, audibly, and/or tactilely prompting the user to pay attention to incorrect actions or an incorrect force applying manner. In other embodiments, the alarm device may also be disposed on the G sensors 120-122 and/or the EMG sensors 130-132 for alarming the user, which is not limited herein.

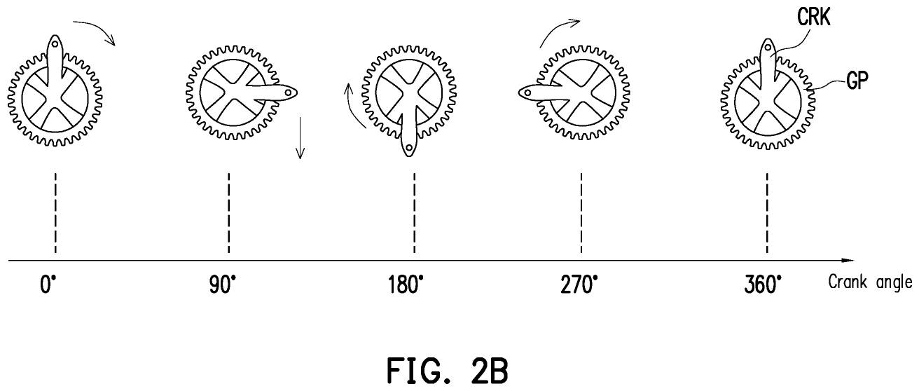

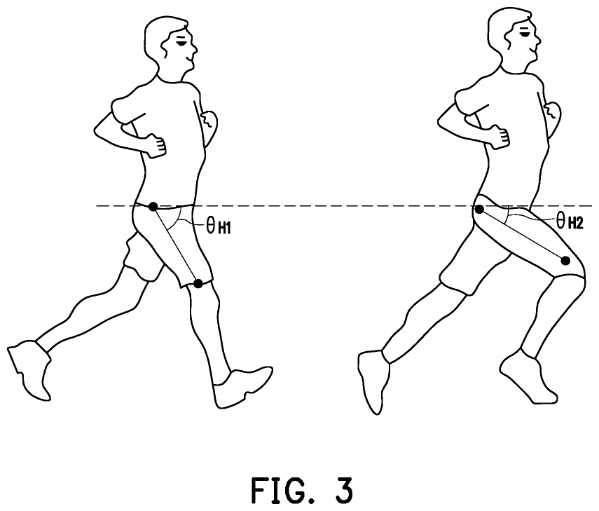

Various use scenarios of the action monitoring system 100 of the embodiments of the disclosure will be described below. Taking a cycling sport as an example, the action monitoring system 100 may use, for example, only two G sensors (for example, the G sensors 120-121) which are respectively disposed on the hip and the knee of the human body and EMG sensors (for example, the EMG sensors 130-132) which are respectively disposed on the muscle parts between the hip and the knee of the human body, such as quadriceps, biceps femoris and hip muscle groups. FIG. 2A is a flowchart illustrating a method for action monitoring of a reciprocating sport according to another embodiment of the disclosure. Referring to FIG. 1A and FIG. 2A simultaneously, the present embodiment is adapted for the action monitoring system 100 illustrated in FIG. 1A and includes steps as follows.

First, in step S210, in a process of a cycling sport, the calculation apparatus 110 may calculate a relative angle of the hip and the knee by using the G sensors 120 and 121 disposed on the hip and the knee based on a horizontal line and sense a startup sequence of the muscle parts by using the EMG sensors 130-132 disposed on the muscle parts among the hip, the knee and the ankle.

Then, in step S220, the calculation apparatus 110 may estimate a crank angle that one of the feet of the human body steps on a pedal of a pedaling apparatus according to the calculated relative angle.

In an embodiment, the calculation apparatus 110 of the present embodiment may first test actions performed by the user to ride a bike before the user does the cycling sport. For example, the calculation apparatus 110 may request the user to ride the bike for a period of time and collect information related to relative angles of the hip and the knee, current crank angles and so on when the user performs various stepping/lifting actions on the pedals of the bike in the process of cycling, so as to record the collected information in the storage device of the calculation apparatus 110 or upload the collected information to the remote server for subsequent look-up and comparison. In this way, when the user actual rides the bike, the calculation apparatus 110 may estimate the crank angle that the foot of the human body steps on the pedal of the pedaling apparatus according to the relative angle of the hip of the knee of the human body which is sensed at that time by looking up in the storage device (or the remote server).

In another embodiment, the calculation apparatus 110 may also first obtain specifications of the bike (for example, a dimension and a structure of each component of the bike) and collect relative positions of the ankle (which represents a position of the pedal) with respect to the hip (which represents a position of the seat) that the user implements various stepping/lifting actions on the bike pedal during the period of testing the user's actions of cycling, so as to estimate the crank angle according to a geometric relationship between the pedal and the seat recorded in the specifications of the bike. Through recording the collected information recorded in the storage device of the calculation apparatus 110 or uploading the same to the remote server, the crank angle may be subsequently obtained through look-up and comparison.

Thereafter, in step S230, the calculation apparatus 110 may determine the stepping action or the lifting action performed on the pedal by the foot according to the crank angle. In an embodiment, the calculation apparatus 110 may determine whether the estimated crank angle falls within a predetermined angle range (for example, 90°±10°), and if the determination result is yes, it may be determined that the user is performing the stepping action on the pedal with one of the feet (for example, the left foot) and performing the lifting action on the pedal with the other foot (the right foot). In other embodiments, the calculation apparatus 110 may determine whether the estimated crank angle falls within another predetermined angle range (for example, 200°±10°), and if the determination result is yes, it may be determined that the user is performing the lifting action on the pedal with one of the feet (for example, the right foot) and performing the stepping action on the pedal with the other foot (the left foot).

For example, FIG. 2B is a diagram illustrating a relationship between the user's stepping action and lifting action and the crank angle according to the embodiment depicted in FIG. 2A of the disclosure. Referring to FIG. 2B, it illustrates relationships between positions of a gear plate GP and a crank CRK and the stepping action and the lifting action performed by the feet of the human body when the foot of the user steps on or lift the pedal in the present embodiment. When a direction of the crank CRK is horizontally rightward, the crank angle is 90°, and it may be determined in this circumstance that the user is performing the stepping action. When the direction of the crank CRK is vertically downward, the crank angle is 180°, and it may be determined in this circumstance that the user is performing the lifting action. When the direction of the crank CRK is horizontally leftward, the crank angle is 270°, and it may be determined in this circumstance that the user is still performing lifting action. When the direction of the crank CRK is vertically upward, the crank angle is 360°, it may be determined in this circumstance that the user does not perform any action or is ready to perform the next stepping action. By observing or recording the stepping action or the lifting action performed on the pedal by the user, the crank angle corresponding to the stepping or the lifting action performed by the user may be obtained. Accordingly, whenever the crank angle is calculated by the calculation apparatus 110 of the embodiments of the disclosure, the stepping action or the lifting action performed on the pedal by the user may be determined according to the calculated crank angle.

Accordingly, in step S240, the calculation apparatus 110 may obtain reference data related to a startup sequence of muscle parts according to the determined the actions and compare the sensed startup sequence of the muscle parts with the reference data related to the startup sequence of the muscle parts, thereby determining whether a force applied by the human body to perform the actions is correct.

In an embodiment, if the determined actions are the stepping action, the reference data corresponding to the stepping action may be obtained, wherein the startup sequence of the muscle parts may be recorded as the gluteus maximus muscle→the quadriceps muscle, the calculation apparatus 110 may compare the sensed startup sequence of the muscle parts with the reference data, thereby determining whether the force applied by the human body to perform the actions is correct. In other embodiments, if the determined actions are the lifting action, the reference data corresponding to the lifting action may be obtained, wherein the startup sequence of the muscle parts may be recorded as the tibialis anterior muscle→the biceps femoris muscle→the iliopsoas muscle, the calculation apparatus 110 may compare the sensed startup sequence of the muscle parts with the aforementioned reference data, thereby determining whether the force applied by the human body to perform the actions is correct. The reference data related to the startup sequence of the muscle parts corresponding to each action may be previously stored in the storage device (or the remote server).

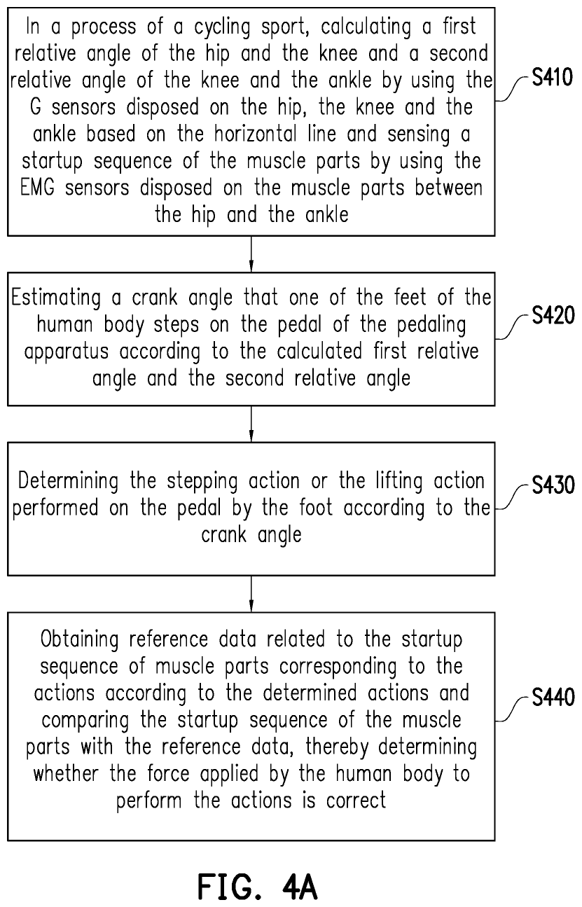

In an embodiment, two G sensors may also be used to determine whether actions involved in a jogging sport are correct. FIG. 3 illustrates an example of a reciprocating sport done by the human body according to another embodiment of the disclosure. Referring to FIG. 1 and FIG. 3 simultaneously, in a process of jogging, the action monitoring system 100 uses, for example, only two G sensors (for example, the G sensors 120-121) which are respectively disposed on the hip and the knee of the human body, EMG sensors (for example, the EMG sensors 130-132) which are respectively disposed on muscle parts of a leg of the human body, wherein the muscle parts may be selected from, for example, a hip muscle group, a quadriceps muscle, a biceps femoris muscle, a calf muscle group, etc. In the present embodiment, the action monitoring system 100 further includes a pressure sensor (not shown) disposed on one of the feet of the human body, and steps of the method of the present embodiment are described as follows.

In the process of jogging, the calculation apparatus 110 may use the G sensors 120 and 121 respectively disposed on the hip and the knee, based on the horizontal line (for example, the dashed line in FIG. 3), to calculate relative angles (i.e., included angles θ_{H1} and θ_{H2} of a connection line between the hip and the knee with respect to the horizontal line as illustrated in FIG. 3) and use the EMG sensors 130-132 disposed on the muscle parts of the leg to sense a startup sequence of the muscle parts of the leg.

The calculation apparatus 110 may use the pressure sensor to sense whether a foot lands. When the pressure sensor senses that the foot lands, the calculation apparatus 110 may determine whether the currently calculated relative angle of the hip and the knee falls within a predetermined angle range (for example, 30°±10°), thereby determining whether the jogging action performed by the human body is correct.

When the foot lands, the calculation apparatus 110 may determine that the jogging action performed by the human body is incorrect at this time if the calculation apparatus 110 determines that the currently calculated relative angle θ_{H1} (for example, 70°) of the hip and the knee of the human body does not fall within the predetermined angle range. In contrast, the calculation apparatus 110 determines that the jogging action performed by the human body is correct at this time if the calculation apparatus 110 determines that the currently calculated relative angle θ_{H2} (for example, 35°) of the hip and the knee of the human body falls within the predetermined angle range.

Besides determining whether the jogging action is correct, the calculation apparatus 110 may also determine whether a force applied by the user to perform the jogging action is correct. For example, the actions of jogging may be divided into four phases, which are respectively a ground contact phase, a stance phase, a propulsion phase and a swing phase. Taking a jogging action performed after the left foot lands for example, in the ground contact phase, a startup sequence of the muscle parts may be the left plantar fascia→the subtalar joint (which is reference data related to a startup sequence of the muscle parts). In the stance phase, a startup sequence of the muscle parts may be the left foot heal tendon→the soleus muscle→the gastrocnemius muscle (which is reference data related to a startup sequence of the muscle parts). In the propulsion phase, a startup sequence of the muscle parts may be the abdominal muscle→the pelvis→the biceps femoris muscles of the feet (which is reference data related to a startup sequence of the muscle parts). In the swing phase, a startup sequence of the muscle parts may be the right biceps femoris muscle→the right rectus femoris muscle (which is the reference data related to a startup sequence of the muscle parts). The calculation apparatus 110 may compare the sensed startup sequence of the muscle parts with the reference data, thereby determining whether the force applied by the human body to perform each action is correct. The reference data related to the startup sequence of the muscle parts corresponding to each action may be previously stored in the storage device (or the remote server).

In an embodiment, when determining that the jogging action performed by the human body is incorrect, the calculation apparatus 110 may execute, for example, an alarming operation to prompt the user that the jogging action being currently performed by the human body is incorrect. For example, the calculation apparatus 110 may be configured with pressure sensors respectively on a foot heel and a foot sole to sense the user lands the foot sole or the foot heel first in the process of jogging. If determining that the user lands the foot heel first in the process of jogging, the calculation apparatus 110 may determine that the user's jogging action is incorrect and execute the alarming operation. If determining that the user lands the foot sole first in the process of jogging, but the relative angle of the hip and the knee does not fall within the predetermined angle range, the calculation apparatus 110 may also determine that the user's jogging action is incorrect and execute the alarming operation.

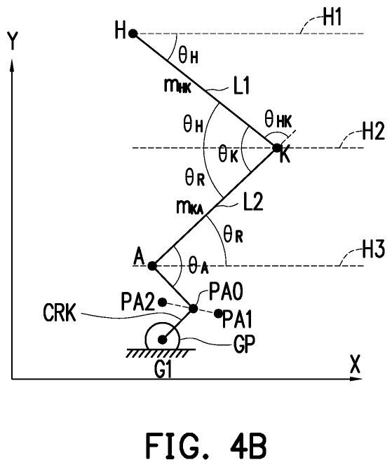

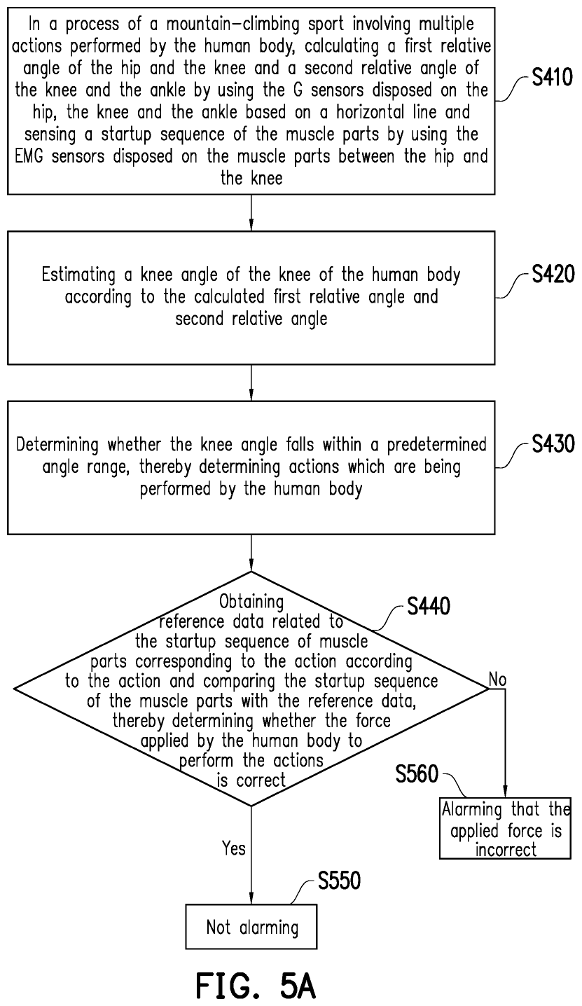

In an embodiment, the action monitoring system 100 may use, for example, three G sensors (for example, the G sensors 120-122) which are respectively disposed on the hip, the knee and the ankle of the human body and three EMG sensors (for example, the EMG sensors 130-132) which are respectively disposed on the muscle parts between the hip and the ankle of the human body. FIG. 4A is a flowchart illustrating a method for action monitoring of a reciprocating sport according to another embodiment of the disclosure. Referring to FIG. 1 and FIG. 4A simultaneously, the method of the present embodiment is adapted for the action monitoring system 100 illustrated in FIG. 1A and includes the following steps.

First, in step S410, in a process of a cycling sport, the calculation apparatus 110 may calculate a first relative angle of the hip and the knee and a second relative angle of the knee and the ankle by using the G sensors 120-122 disposed on the hip, the knee and the ankle based on the horizontal line and sense a startup sequence of the muscle parts by using the EMG sensors 130-132 disposed on the muscle parts between the hip and the ankle.

Thereafter, in step S420, the calculation apparatus 110 may estimate a crank angle that one of the feet of the human body steps on the pedal of the pedaling apparatus according to the calculated first relative angle and the second relative angle.

To be detailed, being similar to the embodiment illustrated in FIG. 2A, the calculation apparatus 110 of the present embodiment may first test actions performed by the user to ride the bike before the user does the cycling sport or alternatively, first obtain the specifications of the bike. The calculation apparatus 110 may request the user to ride the bike for a period of time and collect information related to relative angles of the hip and the knee, current crank angles and so on or relative positions of the ankle with respect to the hip and relationships with crank angles when the user performs various stepping/lifting actions on the bike pedal in the process of cycling, so as to record the collected information in the storage device of the calculation apparatus 110 or upload the collected information to the remote server for subsequent look-up and comparison. In this way, the calculation apparatus 110 may estimate the crank angle that the foot of the human body steps on the pedal of the pedaling apparatus according to the first relative angle of the hip and the knee of the human body and the second relative angle of the knee and the ankle of the human body which are sensed at that time by looking up data stored in the storage device (or the remote server).

FIG. 4B illustrates an example of estimating the crank angle that one of the feet of the human body steps on the pedal of the pedaling apparatus according to the embodiment depicted in FIG. 4A of the disclosure. In the present embodiment, a relative position of the foot of the human body with respect to the pedaling apparatus is as illustrated in in FIG. 4B, wherein a coordinate point H represents a position of the hip, a coordinate point K represents a position of the knee, a line L1 between the coordinate points H and K represents the leg, and a slope m_{HK} of the line L1 is calculated by:

$${m}_{HK} = \frac{{Y}_{H} - {Y}_{K}}{{X}_{H} - {X}_{K}} \qquad ( 1 )$$

Therein, Y_{H} represents a position of the coordinate point H on the Y axis, Y_{K} represents a position of the coordinate point K on the Y axis, X_{H} represents a position of the coordinate point H on the X axis, and X_{K} represents a position of the coordinate point K on the X axis. An angle θ_{H} formed between the line L1 and a horizontal line H1 where the coordinate point H is located is the first relative angle.

On the other hand, the ankle of the human body is located at a position of a coordinate point A, and a line L2 between the coordinate points K and A represents a calf. A slope m_{KA} of the line L2 is calculated by:

$${m}_{KA} = \frac{{Y}_{K} - {Y}_{A}}{{X}_{K} - {X}_{A}} \qquad ( 2 )$$

Therein, Y_{A} represents a position of the coordinate point A on the Y axis, Y_{K} represents a position of the coordinate point K on the Y axis, X_{A} represents a position of the coordinate point A on the X axis, and X_{K} represents a position of the coordinate point K on the X axis. An angle θ_{R} formed between the line L2 and a horizontal line H2 where the coordinate point K is located is the second relative angle, and an angle formed between the line L2 and a horizontal line H3 where the coordinate point A is located is also the second relative angle θ_{R} as well.

Thereafter, the calculation apparatus 110 may utilize the principle of trigonometric function of:

$$tan {θ}_{HK} = \frac{{m}_{HK} - {m}_{KA}}{1 + ( {m}_{HK} * {m}_{KA} )} \qquad ( 3 )$$

to perform an operation of tan⁻¹ on tanθ_{HK} to obtain an angle of θ_{HK}, wherein

θ_{K}=180−θ_{HK} (4)

With Expressions (1) through (4), the calculation apparatus 110 may obtain a knee angle θ_{K} formed between the line L2 and the line L1, which is a sum of the first relative angle θ_{H} and the second relative angle θ_{R}.

By utilizing the first relative angle θ_{H} and the second relative angle θ_{R} and in combination with parameters (for example, relative positions or distances between the bike pedal and the seat) of the previously obtained specifications of the bike, the calculation apparatus 110 may estimate the crank angle that the foot of the human body steps on the pedal of the pedaling apparatus. A dashed line formed by a connection line of coordinate points PA1, PA0 and PA2 represents the pedal, a line from a center point G1 of the gear plate GP to the center point PA0 of the pedal represents the crank CRK, and an angle between such line and a vertical line represents the crank angle.

Returning to the process illustrated in FIG. 4A, in step S430, the calculation apparatus 110 may determine the stepping action or the lifting action performed on the pedal by the foot according to the crank angle. For example, the calculation apparatus 110 may determine whether the estimated crank angle falls within a predetermined angle range (for example, 90°±10°), and if the determination result is yes, it may be determined that the user is performing the stepping action on the pedal with one of the feet (for example, the left foot) and performing the lifting action on the pedal with the other foot (the right foot) at this time. In other embodiments, the calculation apparatus 110 may determine whether the estimated crank angle falls within another predetermined angle range (for example, 200°±10°), and if the determination result is yes, it may be determined that the user is performing the lifting action on the pedal with one of the feet (for example, the right foot) at this time and performing the stepping action on the pedal with the other foot (the left foot).

Accordingly, in step S440, the calculation apparatus 110 may obtain reference data related to a startup sequence of muscle parts corresponding to the actions determined in step S430 and compare the startup sequence of the muscle parts with the reference data, thereby determining whether the force applied by the human body to perform the actions is correct. The implementation of this step that the calculation apparatus 110 determines whether the force applied by the human body to perform the actions is correct is the same as or similar to that of step S240 in the embodiment described above, and thus, the detailed contents will not be repeated.

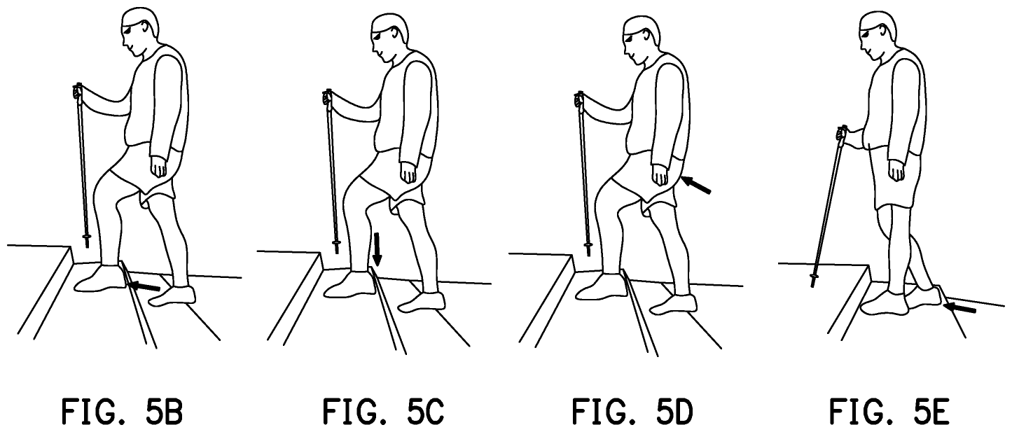

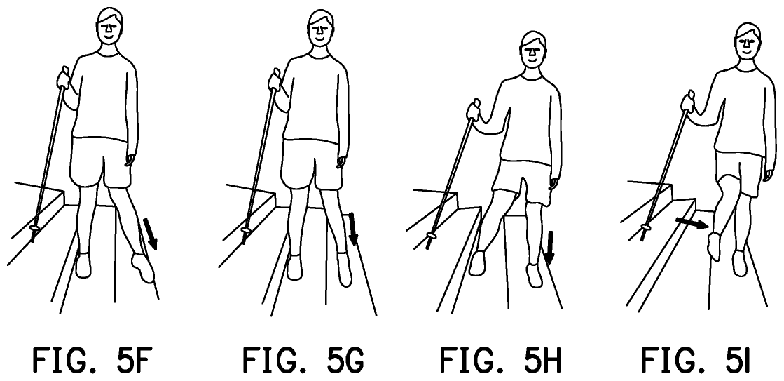

In an embodiment, three G sensors may also be used to sense whether a mountain-climbing sport is correct. FIG. 5A is a flowchart illustrating a method for action monitoring of a reciprocating sport according to another embodiment of the disclosure. Referring to FIG. 1 and FIG. 5A simultaneously, in a process of a mountain-climbing sport, the action monitoring system 100 may use, for example, three G sensors (for example, the G sensors 120-122) which are respectively disposed on the hip, the knee and the ankle of the human body and three EMG sensors (for example, the EMG sensors 130-132) which are respectively disposed on the muscle parts of the leg of the human body, wherein the muscle parts may be selected from a hip muscle group, a quadriceps muscle, a biceps femoris muscle, a calf muscle group, etc., and steps of the method of the present embodiment are described as follows.

In step S510, in a process of a mountain-climbing sport involving multiple actions performed by the human body, the calculation apparatus 110 may calculate a first relative angle of the hip and the knee and a second relative angle of the knee and the ankle by using the G sensors 120-122 disposed on the hip, the knee and the ankle (which may be inferred with reference to the above descriptions related to the first relative angle of the hip and the knee and the second relative angle of the knee and the ankle in step S410) based on a horizontal line and sense a startup sequence of the muscle parts by using the EMG sensors 130-132 disposed on the muscle parts between the hip and the knee.

In step S520, the calculation apparatus 110 may estimate a knee angle of the knee of the human body according to the calculated first relative angle and second relative angle. The manner of estimating the knee angle of the knee of the human body of the present embodiment is the same as or similar to that of the above embodiment illustrated in FIG. 4B, and thus, the detailed contents will not be repeated.

Thereafter, in step S530, the calculation apparatus 110 may determine whether the knee angle falls within a predetermined angle range, thereby determining the actions which are being performed by the human body. In an embodiment, the calculation apparatus 110 may determine whether the estimated knee angle is continuously reduced from 180° and falls within a predetermined angle range (for example, 155°±10°), and if the determination result is yes, it may be determined that the user is performing an uphill or downhill action (or an action upstairs or downstairs) at this time. In other embodiments, the calculation apparatus 110 may determine whether the estimated knee angle is continuously reduced from 180° and falls within a predetermined angle range (for example, 175°±10°), and if the determination result is yes, it may be determined that the user is performing a walking action at this time.

Accordingly, in step S540, the calculation apparatus 110 may obtain reference data related to a startup sequence of muscle parts corresponding to the actions determined in step S530 and compare the startup sequence of the muscle parts with the reference data, thereby determining whether the force applied by the human body to perform the actions is correct. Namely, according to whether the determined actions are the uphill or downhill action or the walking action, the calculation apparatus 110 may look up a corresponding correct startup sequence of the muscle parts to compare it with the sensed startup sequence of the muscle parts, thereby determining whether the force applied by the human body to perform the actions is correct. The reference data related to the corresponding correct startup sequence of the muscle parts may be previously stored in the storage device (or the remote server). If determining that the force applied by the human body to perform the actions is correct, the calculation apparatus 110 enters step S550, without alarming. Otherwise, if determining that the force applied by the human body to perform the actions is incorrect, the calculation apparatus 110 enters step S560 to alarm that the applied force is incorrect or prompt the user to correct the force applying manner by the alarm device.

For example, FIG. 5B through FIG. 5E illustrate an example that the human body performs the uphill action in the mountain-climbing sport according to the embodiment depicted in FIG. 5A of the disclosure. For convenience of illustration, the human body starting the uphill action with the left foot is taken as an exemplary example in the present embodiment, however, the present embodiment does not limit the uphill action to be started with the left foot or the right foot and may be performed by alternating between the left foot and the right foot.

In the process of the uphill action, as illustrated in FIG. 5B, the user may step on a front step with the left foot, and the startup sequence of the muscle parts at this time may be the left gluteus maximus muscle→the left quadriceps muscle→the left biceps femoris muscle. Thereafter, as illustrated in FIG. 5C, the left foot of the user may apply a force downward to firmly step on the step, and the startup sequence of the muscle parts at this time may be the left quadriceps muscle→the left foot sole. When the user shifts the center of gravity (i.e., shifts the center of gravity from the left foot to the right foot), as illustrated in FIG. 5D, the startup sequence of the muscle parts at this time may be the left gastrocnemius muscle→the left quadriceps muscle→the left biceps femoris muscle. Finally, when the user completes the shift of the center of gravity and subsequently steps on the step with the right foot, as illustrated in FIG. 5E, the startup sequence of the muscle parts at this time may be the right tibialis anterior muscle→the right a biceps femoris muscle→the right foot sole. Accordingly, the human body may perform a complete uphill action by completing the actions illustrated in FIG. 5B through FIG. 5E.

On the other hand, FIG. 5F through FIG. 5I illustrate an example that the human body performs the downhill action in the mountain-climbing sport according to the embodiment depicted in FIG. 5A of the disclosure. The human body starting the downhill action with the left foot is also taken as an exemplary example in the present embodiment, however, the present embodiment does not limit the downhill action to be started with the left foot or the right foot and may be performed by alternating between the left foot and the right foot.

In the process of the downhill action, as illustrated in FIG. 5F, the user may first move the left foot sideward to a lower step, and the startup sequence of the muscle parts at this time may be the right quadriceps muscle→the right gastrocnemius muscle→the left gluteus maximus muscle→the left tibialis anterior muscle. Thereafter, as illustrated in FIG. 5G, the left foot of the user may step on the lower step, and the startup sequence of the muscle parts at this time may be the left gastrocnemius muscle→the left foot sole. Thereafter, the left foot of the user may apply a force downward to firmly step on the step, as illustrated in FIG. 5H, and the startup sequence of the muscle parts at this time may be the left gastrocnemius muscle→the left quadriceps muscle→the left tibialis anterior muscle. Finally, the user subsequently may step the right foot on the lower step, as illustrated in FIG. 5I, and the startup sequence of the muscle parts at this time may be the left quadriceps muscle→the left gastrocnemius muscle→the right gluteus maximus muscle→the right quadriceps muscle→the right tibialis anterior muscle→the right foot sole.

According to the startup sequences of the muscle parts of the uphill and the downhill actions, the calculation apparatus 110 may, in step S540, compare the determined startup sequence of the muscle parts corresponding to the uphill or downhill action or the walking action with the currently sensed startup sequence of the muscle parts, thereby determining whether the force applied by the user to perform the uphill or downhill action is correct.

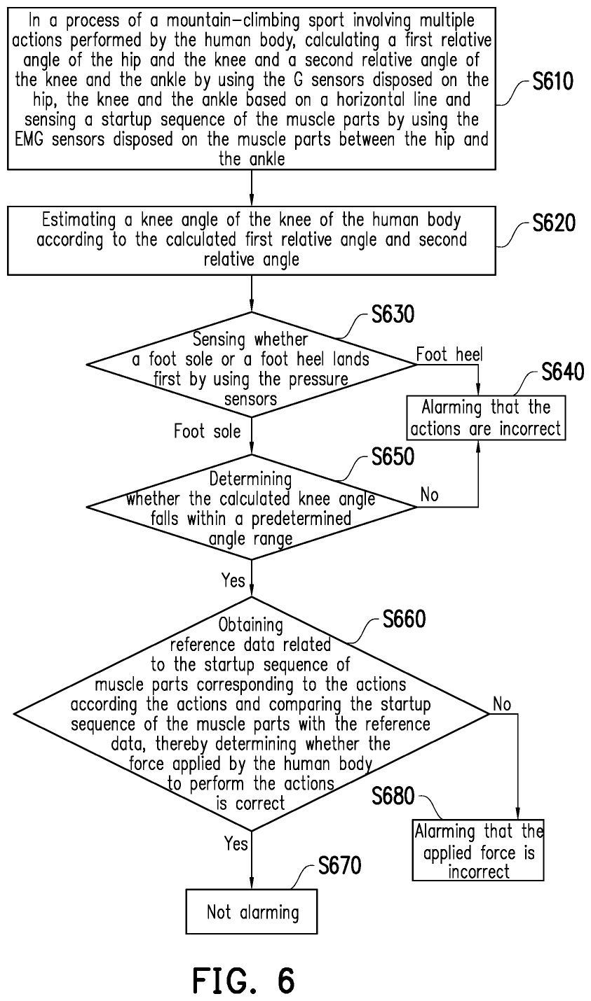

In an embodiment, the implementation manner of determining whether the actions and the force applied by the human body to perform the actions is correct according to the knee angle may further be combined with the sensing of pressure sensors, thereby simultaneously determining whether the actions and the force applied by the human body to perform the actions are correct. For example, FIG. 6 is a flowchart illustrating a method for action monitoring of a reciprocating sport according to another embodiment of the disclosure. Referring to FIG. 1 and FIG. 6 synchronously, in the present embodiment, the action monitoring system 100 further includes pressure sensors disposed on the foot sole and the foot heel of the human body, and steps of the method of the present embodiment are described as follows.

In step S610, in a process of a mountain-climbing sport, the calculation apparatus 110 may calculate a first relative angle of the hip and the knee and a second relative angle of the knee and the ankle by using the G sensors 120-122 disposed on the hip, the knee and the ankle based on a horizontal line and senses a startup sequence of muscle parts by using the EMG sensors 130-132 disposed on the muscle parts between the hip and the ankle.

In step S620, the calculation apparatus 110 may estimate a knee angle of the knee of the human body according to the calculated first relative angle and second relative angle. The implementation manner of estimating the knee angle of the human body of the present embodiment is the same as or similar to that of the embodiment illustrated in FIG. 4B, and thus, the detailed contents will not be repeated.

Thereafter, in step S630, the calculation apparatus 110 may sense whether the foot sole or the foot heel lands first by using the pressure sensors in the process of the uphill or downhill action in the mountain-climbing sport performed by the human body. Because the manner that the foot heel lands first may cause greater impact on the knee of the human body and may result in sports injuries, if sensing that the foot heel lands first, in step S640, the calculation apparatus 110 may determine that the actions are incorrect and alarm that the actions are incorrect by using the alarm device. In contrast, if sensing that the foot sole lands first, the calculation apparatus 110 may, in step S650, further determine whether the calculated knee angle falls within a predetermined angle range, thereby determining whether the actions performed by the human body is correct. The calculation apparatus 110 may not only determine the actions performed by the human body according to, for example, step S530 of the above embodiment illustrated in FIG. 5A, but also determine whether the calculated knee angle falls within the predetermined angle range, thereby determining whether the actions performed by the human body is correct. For example, whether the uphill action performed by the user is correct may be determined according to whether the knee angle falls within a predetermined angle range (for example, 155°±10°). If determining that the actions are incorrect, in step S640, the calculation apparatus 110 may alarm that the actions are incorrect by using the alarm device. In contrast, if determining that the actions are correct, the calculation apparatus 110 may, in step S660, obtain reference data related to a startup sequence of muscle parts according the actions determined in step S650 and compare the sensed startup sequence of the muscle parts with the reference data, thereby determining whether the force applied by the human body to perform the actions is correct. If determining that the force applied by the human body to perform the actions is correct, the calculation apparatus 110 enters step S670, without alarming. Otherwise, if determining that the force applied by the human body to perform the actions is incorrect, the calculation apparatus 110 enters step S680 to alarm that the applied force is incorrect by using the alarm device, thereby reminding the user to correct the force applying manner. The implementation manners of steps S660 through S680 are the same as or similar to those of steps S540 through S560, and thus, the detailed contents will not be repeated.

Based on the above, the method and the system for action monitoring of the reciprocating sports according to the embodiments of the disclosure can determine whether each action is correct and whether the force applied to perform each action is correct according to the data sensed by the G sensors and the EMG sensors in the process of the sports, such as cycling, jogging, mountain-climbing, stepping, hiking, etc., performed by the user, so as to remind the user to correct the actions or the force applying manner by means of alarming. In this way, the probability of the occurrence of sports injuries can be reduced, and the sports efficiency can be improved.

It will be apparent to those skilled in the art that various modifications and variations can be made to the structure of the disclosed embodiments without departing from the scope or spirit of the disclosure. In view of the foregoing, it is intended that the disclosure cover modifications and variations of this disclosure provided they fall within the scope of the following claims and their equivalents.

What is claimed is:

1. A method for action monitoring of a reciprocating sport, adapted for a monitoring system comprising a calculation apparatus, at least one gravity sensor (G sensor) and at least one electromyography sensor (EMG sensor), wherein the at least one G sensor is disposed on at least one motion part of a human body, and the at least one EMG sensor is disposed on at least one muscle part of the human body, the method comprising:
in a process of the reciprocating sport involving multiple actions performed by the human body, sensing a relative angle of each of the at least one motion part with respect to a reference position of the human body by the at least one G sensor and sensing a startup sequence of the at least one muscle part by the at least one EMG sensor;
determining the actions performed by the human body according to the sensed relative angle of each of the at least one motion part; and
determining whether a force applied by the human body to perform the actions is correct according to the actions and the startup sequence of the at least one muscle part.

2. The method for action monitoring according to claim 1, wherein the step of determining whether the force applied by the human body to perform the actions is correct according to the actions and the startup sequence of the at least one muscle part comprises:
obtaining reference data related to a startup sequence of at least one muscle part of the actions according to the actions and comparing the startup sequence of the at least one muscle part with the reference data to determine whether the force applied by the human body to perform the actions is correct.

3. The method for action monitoring according to claim 1, wherein the step of sensing the relative angle of each of the at least one motion part with respect to the reference position of the human body by the at least one G sensor and sensing the startup sequence of the at least one muscle part by the at least one EMG sensor comprises:
calculating a relative angle of a hip and a knee by the G sensors disposed on the hip and the knee and accordingly determining the actions performed by the human body; and
sensing the startup sequence of the at least one muscle part by the at least one EMG sensor disposed on the at least one muscle part between the hip and the knee.

4. The method for action monitoring according to claim 3, wherein the step of calculating the relative angle of the hip and the knee by the G sensors disposed on the hip and the knee and accordingly determining the actions performed by the human body comprises:
estimating a crank angle that a foot of the human body steps on a pedal of a pedaling apparatus according to the calculated relative angle; and
determining a stepping action or a lifting action performed on the pedal by the foot according to the crank angle.

5. The method for action monitoring according to claim 3, wherein the monitoring system further comprises a pressure sensor disposed on a foot of the human body, and the step of calculating the relative angle of the hip and the knee by the G sensors disposed on the hip and the knee and accordingly determining the actions performed by the human body comprises:
sensing whether the foot lands by the pressure sensor; and
determining whether the calculated relative angle falls within a predetermined angle range when the pressure sensor senses that the foot lands, and accordingly determining whether the actions performed by the human body are correct.

6. The method for action monitoring according to claim 1, wherein the step of sensing the relative angle of each of the at least one motion part with respect to the reference position of the human body by the at least one G sensor and sensing the startup sequence of the at least one muscle part by the at least one EMG sensor comprises:
calculating a first relative angle of a hip and a knee and a second relative angle of the knee and an ankle by the G sensors disposed on the hip, the knee and the ankle and accordingly determining the actions performed by the human body; and
sensing the startup sequence of the at least one muscle part by the at least one EMG sensor disposed on the at least one muscle part between the hip and the ankle.

7. The method for action monitoring according to claim 6, wherein the step of calculating the first relative angle of the hip and the knee and the second relative angle of the knee and the ankle by the G sensors disposed on the hip, the knee and the ankle and accordingly determining the actions performed by the human body comprises:
estimating a crank angle that a foot of the human body steps on a pedal of a pedaling apparatus according to the calculated first relative angle and second relative angle; and
determining a stepping action or a lifting action performed on the pedal by the foot according to the crank angle.

8. The method for action monitoring according to claim 6, wherein the step of calculating the first relative angle of the hip and the knee and the second relative angle of the knee and the ankle by the G sensors disposed on the hip, the knee and the ankle and accordingly determining the actions performed by the human body comprises:
estimating a knee angle of the knee according to the calculated first relative angle and second relative angle; and
determining whether the knee angle falls within a predetermined angle range to determine the actions performed by the human body.

9. The method for action monitoring according to claim 8, wherein the monitoring system further comprises a pressure sensor disposed on at least one of a foot sole and a foot heel of the human body, and the step of determining whether the knee angle falls within the predetermined angle range, and accordingly determining the actions performed by the human body comprises:
sensing whether the foot sole or the foot heel lands first by the pressure sensor;
determining that the actions performed by the human body are incorrect if the foot heel lands first; and
determining whether the calculated knee angle falls within the predetermined angle range if the foot sole lands first, and accordingly determining whether the actions performed by the human body are correct.

10. A system for action monitoring of a reciprocating sport, comprising:
at least one gravity sensor (G sensor), disposed on at least one motion part of a human body;
at least one electromyography sensor (EMG sensor), disposed on at least one muscle part of the human body; and
a calculation apparatus, communicating with the at least one G sensor and the at least one EMG sensor and in a process of the reciprocating sport involving multiple actions performed by the human body, being configured to:
sense a relative angle of each of the at least one motion part with respect to a reference position of the human body by the at least one G sensor and sense a startup sequence of the at least one muscle part by the at least one EMG sensor;
determine the actions performed by the human body according to the sensed relative angle of each of the at least one motion part; and
determine whether a force applied by the human body to perform the actions is correct according to the actions and the startup sequence of the at least one muscle part.

11. The system for action monitoring according to claim 10, wherein the calculation apparatus comprises:
obtaining reference data related to a startup sequence of at least one muscle part of the actions according to the actions and comparing the startup sequence of the at least one muscle part with the reference data to determine whether the force applied by the human body to perform the actions is correct.

12. The system for action monitoring according to claim 10, wherein the calculation apparatus comprises:
calculating a relative angle of a hip and a knee by the G sensors disposed on the hip and the knee and accordingly determining the actions performed by the human body; and
sensing the startup sequence of the at least one muscle part by the at least one EMG sensor disposed on the at least one muscle part between the hip and the knee.

13. The system for action monitoring according to claim 12, wherein the calculation apparatus comprises:
estimating a crank angle that a foot of the human body steps on a pedal of a pedaling apparatus by using the calculated relative angle; and
determining a stepping action or a lifting action performed on the pedal by the foot according to the crank angle.

14. The system for action monitoring according to claim 12, wherein the system for action monitoring further comprises:
a pressure sensor, communicating with the calculation apparatus, wherein the pressure sensor is disposed on a foot of the human body, wherein
the calculation apparatus further comprises:
sensing whether the foot lands by the pressure sensor; and
determining whether the calculated relative angle falls within a predetermined angle range when the pressure sensor senses that the foot lands, and accordingly determining whether the actions performed by the human body are correct.

15. The system for action monitoring according to claim 10, wherein the calculation apparatus comprises:
calculating a first relative angle of a hip and a knee and a second relative angle of the knee and an ankle by the G sensors disposed on the hip, the knee and the ankle and accordingly determining the actions performed by the human body; and
sensing the startup sequence of the at least one muscle part by the at least one EMG sensor disposed on the at least one muscle part between the hip and the ankle.

16. The system for action monitoring according to claim 15, wherein the calculation apparatus comprises:
estimating a crank angle that a foot of the human body steps on a pedal of a pedaling apparatus according to the calculated first relative angle and second relative angle; and
determining a stepping action or a lifting action performed on the pedal by the foot according to the crank angle.

17. The system for action monitoring according to claim 15, wherein the calculation apparatus comprises:
estimating a knee angle of the knee according to the calculated first relative angle and second relative angle; and
determining whether the knee angle falls within a predetermined angle range to determine the actions performed by the human body.

18. The system for action monitoring according to claim 17, wherein the system for action monitoring further comprises:
a pressure sensor, communicating with the calculation apparatus, wherein the pressure sensor is disposed on at least one of a foot sole and a foot heel of the human body, wherein the calculation apparatus further comprises:
sensing whether the foot sole or the foot heel lands first by the pressure sensor;
determining that the actions performed by the human body are incorrect if the foot heel lands first; and
determining whether the calculated knee angle falls within the predetermined angle range if the foot sole lands first, and accordingly determining whether the actions performed by the human body are correct.

19. The system for action monitoring according to claim 17, wherein the calculation apparatus further comprises:
determining whether the knee angle falls within the predetermined angle range to determine whether the actions performed by the human body are correct; and
if the actions performed by the human body are determined as incorrect, alarming that the actions are incorrect.

20. The system for action monitoring according to claim 10, wherein the calculation apparatus further comprises:
if the force applied by the human body to perform the actions is determined as incorrect, alarming that the force applied to perform the actions is incorrect.
